(19) United States
(12) Patent Application Publication
(10) Pub. No.: US 2024/0366904 A1
(43) Pub. Date: Nov. 7, 2024

(54) VENTILATION HOSE AND METHOD FOR PRODUCING A VENTILATION HOSE

(71) Applicant: Loewenstein Medical Technology S.A., Luxembourg (LU)

(72) Inventor: Willy Schulze, Roethenbach an der Pegnitz (DE)

(21) Appl. No.: 18/652,537

(22) Filed: May 1, 2024

(30) Foreign Application Priority Data
May 5, 2023 (DE) 102023001816.3
May 24, 2023 (DE) 102023002102.4

(51) Int. Cl. A61M 16/08; B29C 48/00; B29C 48/11; B29C 48/16; B29D 23/18; B29K 27/06; B29K 83/00; B29L 23/00

(52) U.S. Cl. CPC A61M 16/0875; B29C 48/0018; B29C 48/022; B29C 48/11; B29C 48/16; B29D 23/18; A61M 2207/00; B29K 2023/06; B29K 2027/06; B29K 2083/00; B29L 2023/007

(57) ABSTRACT

A method for producing a ventilation hose and a ventilation hose having a continuous wall, which delimits an internal lumen, converted in an extrusion and thermoforming process via thermoplastic and elastic processes from an original mold in a forming process. The lumen is designed to guide respiratory gas, a helix surrounding the wall in a spiral shape on the side facing away from the lumen. The wall is produced from a first material and the helix is produced from a second material, with the helix being connected to the wall.

### CROSS-REFERENCE TO RELATED APPLICATIONS

The present application claims the priorities under 35 U.S.C. § 119 of German Patent Application No. 102023001816.3, filed May 5, 2023, and German Patent Application No. 102023002102.4, filed May 24, 2023, the entire disclosures of which are expressly incorporated by reference herein.

### BACKGROUND OF THE INVENTION

### 1. Field of the Invention

The present invention relates to improved ventilation hoses.

### 2. Discussion of Background Information

Ventilation hoses are an important component of medical ventilation. Medical ventilation is used in patients having difficulties in breathing. Some important factors which are to be taken into consideration in ventilation hoses: ventilation hoses are to be flexible in order to adapt themselves to the movements of the patient and to make the intubation as pleasant as possible in this way, because in the event of movement changes or sudden movements, the resulting force actions do not act directly or in an undiminished manner on the body of the patient. The size of the ventilation hose is important in the case of invasive medical ventilation in order to ensure that it matches with the diameter of the airways of the patient and ensures an adequate air supply.

Ventilation hoses are to be produced from a material which is safe for the patient and does not trigger allergies. Hoses are typically produced, for example, from TPE, silicone, or PVC. Most ventilation hoses consist of flexible PVC material. The properties of the material have to be taken into consideration in the selection of the materials, such as the flexibility, the strength, and the chemical resistance. The PVC materials are brought by an extrusion method into the desired hose diameter and hose length. The material is pressed through a die and extruded onto a shaft, which is responsible for the internal shaping and for the winding and molding of the hose, and is formed by cooling into a solid hose. Good leak-tightness of the ventilation hose is crucial to ensure that the patient receives sufficient oxygen and no air escapes. Ventilation hoses are to have fittings which enable them to be connected securely to the ventilator or the breathing mask of the patient. The length of the hose is important to ensure that it is long enough to be connected comfortably, but not so long that it causes unnecessary entanglements or twists, which could throttle or even interrupt the required oxygen supply. Ventilation hoses can be sterile in order to avoid infections in the patient. However, ventilation hoses are generally not sterile, which reduces the production costs. Moreover, ventilation hoses are to be well marked in order to facilitate the positioning in the body of the patient and ensure safe application. Hoses according to the prior art are flexible and yielding within certain limits.

In view of the foregoing, it would be advantageous to have available an improved hose for medical ventilation, and in particular, a hose that is more comfortable and at the same time more reliable.

### SUMMARY OF THE INVENTION

The invention provides a hose and a method of producing same as set forth in the claims. Further advantages and features result from the general description and the description of the exemplary embodiments.

While the hose of the present invention is flexible, it is desirably also less yielding than versions from the prior art. If a hose of the prior art is compared to the hose of the present invention, it is shown that the hose of the prior art is more easily expanded both axially and radially.

The hose according to the invention preferably offers many advantages. One significant advantage may be offered by a ventilation hose having a continuous wall which delimits an internal lumen, wherein the lumen is designed for guiding respiratory gas, wherein a helix encloses the wall on the side facing away from the lumen in a spiral shape, wherein the wall is produced from a first material and wherein the helix is produced from a second material, wherein the helix is connected to the wall. The helix and the wall can also be produced from different materials.

In all embodiments, it is particularly advantageous and preferred for the helix to have a lower side and to be connected by means of this lower side directly and permanently to the outer wall.

It is also advantageous and preferred for the helix to have a rounded outer contour in cross section and to taper from the lower side to an upper side in the direction of a maximum helix height.

The helix height ranges from about 1.5 mm to about 3 mm, preferably about 2.3 mm, measured perpendicularly pointing away from the outer wall.

In all embodiments, it is also particularly advantageous and preferred for the width of the helix (on the lower side which rests on the outer wall) to range from about 1.8 mm to about 4 mm, preferably from about 2.3 mm to about 3.3 mm, and particularly preferably about 2.9 mm+/−0.4 mm.

In one advantageous embodiment, the spacing between two helices corresponds to the pitch of the helix, wherein the pitch preferably ranges from about 2 mm to about 8 mm, preferably from about 3 mm to about 7 mm, particularly preferably from about 5 mm+/−0.5 mm. The pitch is determined directly on the outer wall where the helix rests.

It is also advantageous and preferred for the helix to taper with increasing distance from the outer wall, due to which the spacing between two adjacent helices is different at at least three points and wherein the spacing between two adjacent helices is greatest in the area of the maximum helix height.

In all embodiments, it is particularly advantageous and preferred for the leakage of the hose at a respiratory gas pressure of 60 hPa to be less than about 15 ml/min/m, preferably less than about 12 ml/min/m, and especially preferably less than about 10 ml/min/m.

A leak in the ventilation hose can be very dangerous, since it impairs the effectiveness of the ventilation and can result in an inadequate respiratory gas supply for the patient.

If a leak is suspected in the ventilation hose, the hose is to be exchanged immediately. However, before the exchange of the hose, the patient is to be switched over to an alternative respiratory gas supply, such as manual ventilation, in order to ensure that the patient is still adequately supplied with oxygen.

It is also important to check the hoses and all other ventilators for possible leaks or other problems regularly in order to ensure the safety of the patient.

A hose leak relates to a leaky point or a hole in a hose, through which the respiratory gas can escape.

Hose leaks can have various causes, such as wear, damage by sharp objects, inadequate fastening, pressure changes, aging of the material, and many others. In particular, however, an unsuitable material can already have such a leak even without damage, so that ventilation using a hose made of this unsuitable material endangers supply to the patient.

It is also advantageous and preferred for the spring rate to be in the range 25 N/m or greater than about 25 N/m, preferably greater than about 27 N/m, and especially preferably greater than about 30 N/m.

The spring rate relates to the rigidity or elasticity of the hose material. The higher the spring rate, the more rigid the hose is and the more difficult it is to bend or deform it. A lower spring rate means higher flexibility and easier handling of the hose. The selection of the correct spring rate is dependent on the requirements of the application and can vary from producer to producer.

A hose having a low spring rate is generally more flexible and may be bent more easily than a hose having a higher spring rate. It can thus be adapted better to various shapes and contours, which can be advantageous in many applications.

A further advantage of a low spring rate is that the hose is generally less susceptible to cracks and damage, since it can adapt itself better in the event of pressure changes and movements and thus less tension occurs.

However, a hose having an excessively low spring rate can also become unstable and easily deformable, which can result in problems depending on the application. It is therefore important to select the correct spring rate for the respective application.

The spring rate of a ventilation hose relates to the rigidity or resistance of the helix within the hose. A higher spring rate means that more pressure is required to expand the hose, while a lower spring rate means that less pressure is required. The selection of the correct spring rate is dependent on various factors, such as the pressure and volume of the gas that flows through the hose and the requirements for the ventilation of the patient.

A high spring rate in a hose typically means, when more rigid materials are used, that the hose is more rigid and more resistant to deformations.

In the present invention, the wall thickness of the membrane is similarly thick as a significantly less flexible hose. The flexibility is created via the geometry of the helix.

This can have various advantages, depending on the application of the hose:

Reduced expansion: When the hose is under pressure, it expands. A high spring rate means that the hose expands less, which minimizes the pressure loss and increases the efficiency of the medical ventilation.

Precise control: A rigid hose enables more precise control of respiratory gases. This is especially important for specifying certain pressures, flows/flow speeds, or volumes.

Hoses having a high spring rate withstand loads longer and are less susceptible to material fatigue. This can lengthen the service life of the hose.

A rigid hose can react faster to changes in the gas flow. This can be advantageous in applications in which patient triggers are evaluated for the control of the ventilation.

However, it is to be noted that an excessively high spring rate can also result in disadvantages, such as an increased probability of cracks or fractures of the hose in the event of excessively high load.

Compliance and spring rate are both properties of a hose but describe different aspects.

Compliance is a measure of the capability of the hose to deform under a given load or pressure change. It is the counterpart of rigidity and is expressed in units of volume per unit of pressure. Compliance can be viewed as the amount of volume which a hose can change per unit of applied pressure. A hose with high compliance deforms easily, while a hose with low compliance resists deformation.

In one advantageous embodiment, the compliance of the hose is less than about 1.0 ml/hPa/m, preferably less than about 0.8, and especially preferably 0.7 or less than about 0.7. In contrast, the spring rate is a measure of the resistance of the hose to deformation under a given load or a given pressure. It is a measure of the force which is required to compress or stretch the hose by a specific length. The spring rate is expressed in units of force per unit of length. A hose having a high spring rate requires more force to deform it than a hose having a low spring rate. The spring rate only takes into consideration a linear tensile or compressive force, in order to compress or stretch the hose. In the case of compliance, the pressure is applied everywhere in the interior of the hose. The geometry of the wall has less influence here than the rigidity and the wall thickness.

In summary, it may be stated that the compliance indicates how much a hose deforms under a given pressure, while the spring rate measures how much force is necessary to deform the hose by a specific length. The greater the spring rate is, the less is the compliance, because the greater rigidity of the hose permits less deformation under pressure at the same time.

The hose is preferably suitable and designed so that the extensibility is in the range from about 3% to about 39%, preferably in the range from about 5% to about 36%, and especially preferably in the range from about 8% to about 30%.

The extensibility of ventilation hoses varies depending on material and producer. In general, ventilation hoses are produced from flexible materials such as TPE, silicone, or PVC in order to assure a certain extensibility and flexibility. The extensibility is often specified by the so-called “elongation factor”, which specifies by how many percent the hose may be elongated before it tears or is damaged.

However, it is important to note that the extensibility of ventilation hoses is not the only factor which should be taken into consideration in the selection of a suitable hose. Other factors such as size, shape, wall thickness, material quality, and compatibility with disinfectants also have to be taken into consideration to select a suitable hose for the medical ventilation. The extensibility of a ventilation hose is dependent on various factors, such as the material from which the hose is produced, the size of the hose, and the type of the load to which it is subjected. In general, ventilation hoses are manufactured from flexible materials such as silicone or polyurethane, which have a certain extensibility in order to ensure a maximum air supply and discharge. The precise extensibility is dependent on the specific construction and production of the hose, however.

In one advantageous embodiment, the wall is produced from a first TPE or silicone or PVC material and the helix is produced from a second TPE or silicone or PVC material.

It is possible and advantageous that the first material and the second material are identical. In the case of TPE, the use can take place in different degrees of hardness.

According to the invention, the hose is also designed so that the weight per unit of length is less than about 1000 g/m, preferably in the range from about 500 g/m to about 1000 g/m, especially preferably in the range from about 550 g/m to about 800 g/m.

The present ventilation hose is also usable for infants, newborns, and premature infants. The respiratory frequency of an infant is very high, so that the respiratory cycle or the time span from the inhalation phase to the exhalation phase is very short. The lungs of a premature infant are small and stiff and require an even higher respiratory frequency, i.e., a shorter, faster cycle. A high flow rate is therefore required during the inspiration or inhalation phase to transfer the desired respiratory gas volume within this short cycle to the lungs of the infant. The higher the peak pressure is, the higher is the flow rate which is required to reach this peak pressure. The exhalation resistance (expiratory counter pressure) also rises with increasing flow rate. During the exhalation phase, the patient has to exhale respiratory gas. It is desirable to minimize the counter pressure within the hose so that the patient does not have to overcome this pressure in an exhausting manner during exhalation (expiratory resistance). It is therefore desirable to minimize the sources of both the expiratory counter pressure and the expiratory resistance.

A high flexibility of the hoses according to the invention is achieved by the unique combination of the thin-walled hose and the support structure.

These are produced, for example, from POLYVINYL CHLORIDE (PVC) or POLYETHYLENE (PE) or POLYPROPYLENE (PP) or POLYESTER or silicone or THERMOPLASTIC ELASTOMER (TPE).

The invention also provides a method for producing a ventilation hose having a continuous wall which delimits an interior lumen in an extrusion process, wherein the lumen is designed for guiding respiratory gas, wherein a helix encloses the wall on the side facing away from the lumen in a spiral shape, wherein the wall is produced from a first material and wherein the helix is produced from a second material, characterized in that the helix is connected to the wall. Helix and wall are produced in a co-extrusion process.

The helix is necessary to provide the hose with a certain stability, for example, against external force action. Helix and wall are preferably made of the same material.

The first step of the method is the co-extrusion of the starting hose. The method steps described hereinafter then take place to make the hose more flexible and extensible.

In the method, in one method step, the hose is preferably heated above the softening temperature of the material, and, in a second method step, the hose is compressed in relation to its original length, and, in a third method step, the hose is cooled until the temperature falls below the solidification temperature of the material.

In the method, the heating and compression take place in direct succession, before the compressed hose is cooled.

In the method, the hose is heated to above about 70° C., preferably to above about 80° C., and mechanically compressed on a block so that the helices touch one another. The hose is then cooled to a temperature of below about 80° C., preferably below about 70° C., before the compression is ended.

The hose is preferably heated from the inside and cooled from the inside, for example, by a gas flow.

In the scope of this patent application, the terms hose and ventilation hose are used synonymously.

The hose according to the invention ensures optimum ventilation due to the following advantages: A transparent hose makes continuous monitoring of the hose system possible. A reinforcing outer helix keeps the hose diameter constant, even when it is bent. The hose is thus flexible and resistant to buckling, so that the risk of a reduction or interruption in ventilation is reduced. A very low compliance ensures a comfortable application. At the same time, soft, leakproof, and flexible fittings ensure a perfect seal. The connectors are integrated directly into the hose in order to ensure tight connections and avoid leaks. The hoses can be extended in length due to the extensibility.

### BRIEF DESCRIPTION OF THE DRAWINGS

The invention will be described in more detail by way of example on the basis of the drawings, in which:

FIG. 1 shows a hose according to the invention,

FIG. 2a shows another hose according to the invention with several features, FIG. 2c showing FIG. 2b shows an enlarged detail of FIG. 2c,

FIG. 2c shows a section through the hose of FIG. 2a along line A-A,

FIG. 2d also shows an enlarged detail of FIG. 2c,

FIG. 3 shows the fundamental structure of a ventilator, and

FIG. 4 is a graph which shows the spring rate and the elongation of an exemplary hose.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

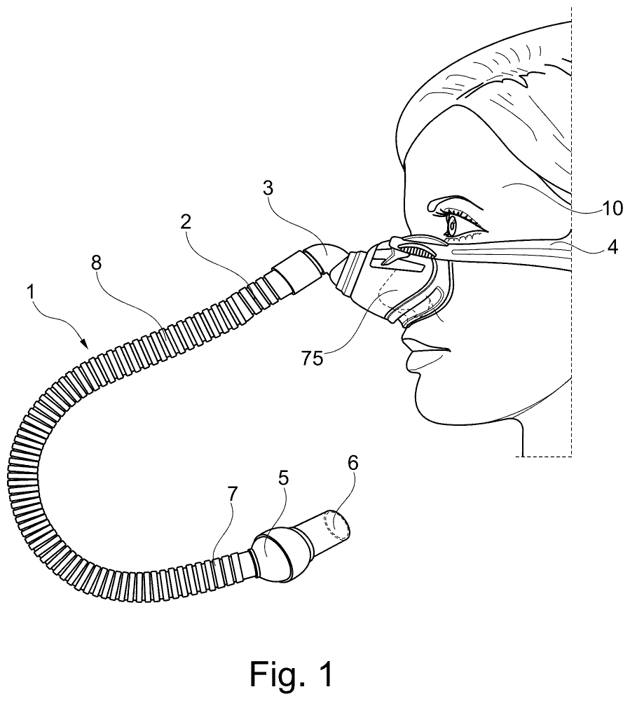

FIG. 1 shows a hose according to the invention. The hose 1 is connected here via a patient interface 75 and a strap 4 to a patient 10. The connection between hose 1 and patient interface 75 is produced via a mask connector 3, which is embodied as curved and having a ball joint here. The mask connector 3 is pushed over the hose end 2 or inserted into the hose end 2. The other hose end is connected to a hose connector 5. The hose connector 5 is embodied here having a ball joint and has a connecting piece 6 at its end facing away from the hose 1, which is used for the connection to another ventilation hose, which has, for example, a larger diameter than the hose 1. Alternatively, the other hose end 7 can be connected to a connecting piece (not shown), which is used for the direct connection to a ventilator.

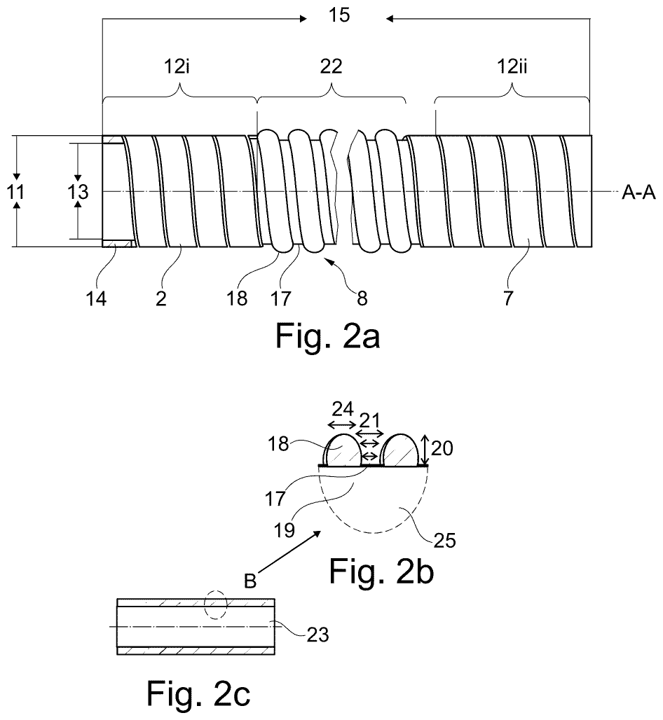

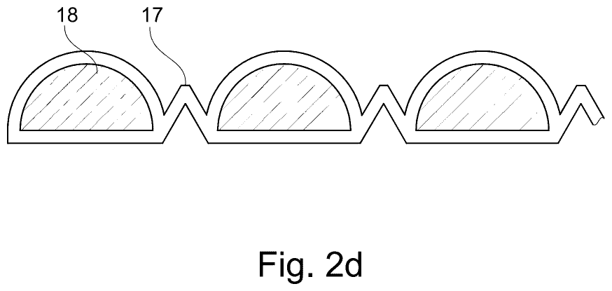

FIG. 2 shows the ventilation hose 1 according to the invention by way of example with several features. The hose has a length 15, which is, for example, 380 to 400 mm, preferably 390 mm. The length can also assume other values, for example, the hose can have a length of 1000 mm or 2000 mm or also 3000 mm or intermediate values thereof. The length 15 is composed of the two hose ends 2, 7 and the hose part 8. The hose ends 2, 7 are shown as equal in length here, however, they can also have different lengths, thicknesses, and shapes. The hose ends 2, 7 are preferably 10-30 mm long here, particularly preferably 20 mm+/−4 mm. The hose part 8 typically makes up the predominant part of the overall length 15, in the present case the hose part 8 without the hose ends is approximately 350 mm long.

As FIG. 1 and FIGS. 2a-d show, the hose is a relatively thin-walled and flexible hose 1 having an external support structure 18, which is embodied as a spiral-shaped helix 18, which extends over the length 15 of the hose along its outer wall 17. The support structure 18 provides the hose 1 with structural strength and rigidity, while it moreover provides flexibility. The provided structural strength and rigidity offer protection from crushing by external forces, helps to avoid a pressure collapse, and reduces the pliability, which is the tendency of a hose to expand or collapse in the event of pressure changes. Although the support structure 18 is preferably spiral-shaped or is arranged around the hose in a spiral shape, other shapes are possible.

FIG. 2c shows a section through the hose along line A-A, partially with a view of the hose interior. The inner surface 23 of the hose can be smooth, which contributes to minimizing both the inspiration flow resistance and the expiration flow resistance. The outer surface of the hose is characterized by the helix 18, which is connected to the outer wall 17.

FIG. 2b shows an enlarged detail of FIG. 2c. The helix 18 is (permanently) connected with a lower side directly to the outer wall 17. The helix 18 extends in a rounded manner from the lower side to the upper side and thus tapers in the direction of the maximum helix height 20. The width 24 of the helix (at the lower side which rests on the outer wall) is in the range 1.8-4 mm, preferably 2.3-3.3 mm, and particularly preferably 2.9 mm+/−0.4 mm. The helix height 20 is in the range 1.5 to 3 mm, preferably 2.3 mm measured perpendicularly pointing away from the outer wall 17.

The spacing between two helices 18 corresponds to the pitch 21. The pitch is preferably in the range 3-8 mm, particularly preferably it is 5 mm+/−0.5 mm. The pitch 21 is determined directly at the outer wall 17, where the helix rests.

Because the helix tapers with increasing distance from the outer wall 17, the spacing between two helices 18 is different at at least three points. The tapering width 24 of the helix 18 contributes to the hose overall being more flexible, since the areas of the helix facing outward only touch late in the event of a strong bend.

FIG. 2d also shows an enlarged detail of FIG. 2c. The helix 18 is connected directly to the outer wall 17 with a lower side. The wall is folded between the helices like a harmonica because of the compression process. The structural basis for the hose is offered in this case in particular by the helix.

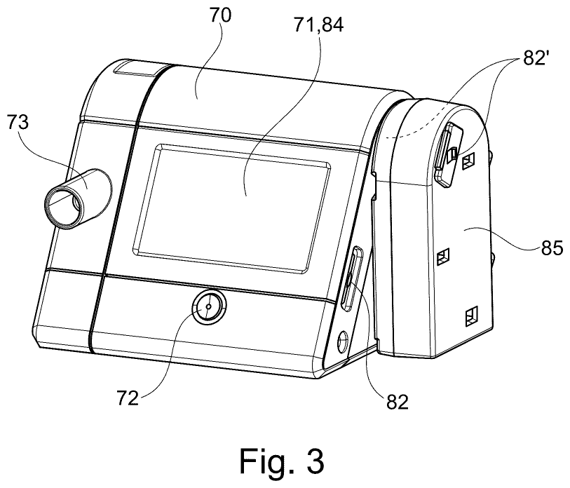

FIG. 3 shows the fundamental structure of a ventilator 70, which is provided with a display device 71, at least one operating element 72, and a hose fitting 73. A patient interface (designed as a breathing mask, for example) 75 is typically connected to the hose fitting 73 via a ventilation hose 74. The patient interface 75 and the ventilation hose 1 for the respiratory gas supply of a patient are not shown. The ventilator 70 has an internal respiratory gas source 76, typically in the form of a fan, and a controller 77 of this respiratory gas source 76. In addition, the ventilator 70 also has a measuring unit 78 and a storage unit 80. The display device 71 is also used as an operating unit 84 of a human-machine interface, typically in the form of a touchscreen display, in addition to the display. The ventilator 70 is typically provided with at least one communication interface 82. The interface 82 is arranged here in a lateral area. The interface 82 is inclined to the horizontal and is located in spatial proximity to the display device 71. The ventilator 70 preferably moreover has a further interface 82, which is used for coupling modules 85. Measuring units in or on the ventilator and/or ventilation hose and/or patient interface are provided for monitoring and controlling flow and pressure, so that the pressure and flow of respiratory gas can be monitored and adapted if necessary, also in consideration of the influences of ventilation hose and/or patient interface on the ventilation.

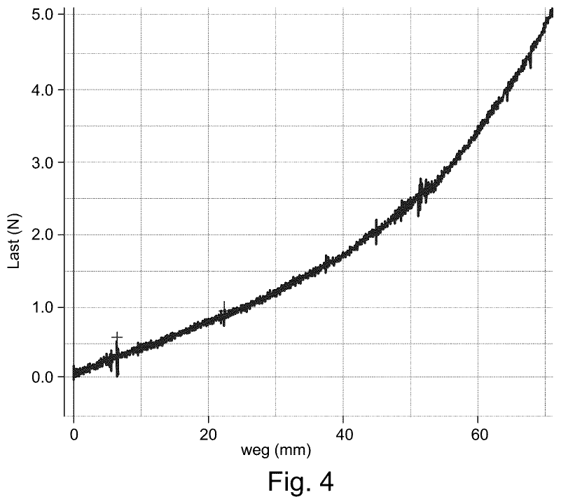

The graph of FIG. 4 shows the spring rate and the elongation of an exemplary hose.

### Calculation of Elongation:

The determination preferably takes place where the nonlinear change of the graph is indicated. The graph rises more vertically there (significant increase of the load at equal distance).

The maximum elongation of the hose by the helix is at this point. When the hose is extended beyond this yield point, it no longer returns to its starting length.

The elongation is thus x mm here.

At a starting length of the hose of y mm
a percentage elongation results, for example, of: 15-30%.

The spring rate is calculated by means of the following formula:

$$R = \frac{F}{δ L}$$

F=the determined force in newtons (N) read from the diagram
δL=the distance covered at the selected force according to the diagram

The following results here:

### Conversion δL of mm in m

$$e . g . \frac{20 mm}{1000 mm / m} = 0.02 m$$

The force is determined at the selected length of, for example, 20 mm on the basis of the diagram.

$$F = 0.75 N$$

Calculation of the helix constant:

$$R = \frac{F}{δ L} = \frac{0.75 N}{0.02 m} = 37.5 N / m$$

The present invention takes into consideration that the pliability, the extent of the expansion of the hoses during a respiratory cycle, is an important design consideration. The basic process will now be described. The pressure and the flow rate through the hose remain essentially equal during the respiratory cycle. In order to ventilate the patient, during the exhalation, a valve is opened on the ventilator, due to which the patient can expel respiratory gas into the hose. This intermittent pressure increase exerts expansion forces on the hose. During the inhalation section of the respiratory cycle, a desired peak pressure has to be achieved at the lungs of the patient in order to deliver a desired respiratory gas volume within the inhalation time span.

The more the hose expands, the more respiratory gas has to be supplied during the inhalation period in order to reach the peak pressure, so that the desired respiratory gas volume is supplied to the lungs of the patient. A lower compliance of the hose is therefore desirable.

Hoses having lower pliability require a lower flow rate to reach the desired peak pressure. Since the flow rate remains constant during the inspiration and expiration phase, hoses having lower compliance during the expiration phase have a lower flow rate. This lower flow rate desirably results in a lower counter pressure against which the patient has to exhale. As a result, the superior low pliability of the present invention reduces the counter pressure in precisely the optimum amount.

The advantageous properties of hoses according to the present invention may be adjusted in the manufacturing process by the selection of material and manufacturing conditions. Alternatively, according to the invention, hoses can also first be manufactured and then modified so that the advantageous properties are achieved.

Previous experiments have shown that the following procedure is suitable in principle:
heat hose (from the inside)
compress hose
cool hose

The hose has to be heated above the softening temperature of the material to achieve a lasting deformation of the wall. This is approximately 70-95° C., preferably 85° C., in the case of the TPE plastic used.

The production takes place, for example, in a sequence of steps;

### Compress Hose

The heated hose has to be compressed so that the properties are changed and retained.

The hose is compressed for this purpose “on block”. The helices adjoin one another here and thus limit the compression.

As soon as the hose has been cooled, the force compressing the hose can be removed.

### Cool Hose

As soon as the temperature of the hose is below the solidification temperature of the material, the deformations-introduced in the heat-remain in existence. With the plastic used, this is a temperature of below 85° C., preferably below 80° C., or even below 70° C.

To sum up, the present invention provides the following items:
1. A ventilation hose, wherein the ventilation hose comprises a continuous wall which delimits an internal lumen, the lumen being configured to guide respiratory gas, a helix enclosing the continuous wall in a spiral shape on the side facing away from the lumen, and the continuous wall being produced from a first material and the helix being produced from a second material, the helix being connected to the continuous wall.
2. The ventilation hose of item 1, wherein the helix has a lower side and is connected by this lower side directly and permanently to the continuous wall.
3. The ventilation hose of at least one of the preceding items, wherein the helix has a rounded outer contour in cross section and tapers from a lower side to an upper side in a direction of the maximum helix height.
4. The ventilation hose of at least one of the preceding items, wherein the width of the helix (at the lower side which rests on the continuous wall) ranges from 1.8 mm to 4 mm, preferably from 2.3 mm to 3.3 mm, and particularly preferably is 2.9 mm+/−0.4 mm.
5. The ventilation hose of at least one of the preceding items, wherein the spacing between two helices corresponds to a pitch of the helix, the pitch preferably ranging from 3 mm to 8 mm, particularly preferably being 5 mm+/−0.5 mm.
6. The ventilation hose of at least one of the preceding items, wherein the helix tapers with increasing distance from the continuous wall, due to which the spacing between two adjacent helices is different at at least three points and wherein the spacing between two adjacent helices is greatest in the area of the maximum helix height.
7. The ventilation hose of at least one of the preceding items, wherein the helix surrounds the continuous wall in the form of a helix of 2.7 mm with 5 mm pitch at an internal diameter of Ø15 mm hose.
8. The ventilation hose of at least one of the preceding items, wherein the continuous wall has a thickness of from 0.05 mm to 0.3 mm, preferably from 0.1 mm to 0.2 mm.
9. The ventilation hose of at least one of the preceding items, wherein the compliance of the hose is less than 1.2 ml/hPa/m, preferably less than 1.0 ml/hPa and especially preferably less than 0.8 ml/hPa or also less than 0.7 ml/hPa.
10. The ventilation hose of at least one of the preceding items, wherein the leakage at a respiratory gas pressure of up to 60 hPa is less than 15 ml/min/m, preferably less than 12 ml/min/m, and particularly preferably less than 10 ml/min/m.
11. The ventilation hose of at least one of the preceding items, wherein the spring rate ranges from 25 N/m or greater than 25 N/m, preferably greater than 28 N/m, and especially preferably greater than 31 N/m.
12. The ventilation hose of at least one of the preceding claims, wherein the extensibility ranges from 3% to 38%, preferably from 5% to 34%, and especially preferably from 8% to 30%.
13. The ventilation hose of at least one of the preceding items, wherein the continuous wall is produced from a first silicone material and the helix is produced from a second PVC or TPE material.
14. The ventilation hose of item 1, wherein the first material and the second material are identical.
15. The ventilation hose of at least one of the preceding items, wherein the weight per unit of length is less than 1000 g/m, preferably from 500/m to 1000 g/m, particularly preferably from 550 g/m to 800 g/m.
16. A method for producing a ventilation hose comprising a continuous wall which delimits an internal lumen, the lumen being configured to guide respiratory gas and a helix enclosing the continuous wall in a spiral shape on the side facing away from the lumen, wherein the method comprises producing the continuous wall from a first material and producing the helix from a second material, and connecting the helix to the continuous wall in a co-extrusion process.
17. The method of item 16, in which in a first method step, the hose is heated above the softening temperature of the material and, in a second method step, the hose is compressed in relation to its original length and, in a third method step, the hose is cooled until its temperature falls below the solidification temperature of the material.
18. The method of any one of items 16 or 17, wherein heating and compression take place in direct succession and the compressed hose is cooled.
19. The method of at least one of items 16 to 18, wherein the hose is heated to above 70° C., preferably to above 80° C. and compressed on block, so that helices touch one another, and wherein the hose is cooled to a temperature of below 80° C., preferably below 70° C., before the compression is ended.
20. The method of at least one of items 16 to 19, wherein the hose is heated from the inside and cooled from the inside.

What is claimed is:

1. A ventilation hose, wherein the ventilation hose comprises a continuous wall which delimits an internal lumen, the lumen being configured to guide respiratory gas, wherein a helix encloses the continuous wall in a spiral shape on a side facing away from the lumen, and wherein the continuous wall is produced from a first material and the helix is produced from a second material, the helix being connected to the continuous wall.

2. The ventilation hose of claim 1, wherein the helix has a lower side and is connected by this lower side directly and permanently to the continuous wall.

3. The ventilation hose of claim 1, wherein the helix has a rounded outer contour in cross section and tapers from a lower side to an upper side in a direction of a maximum helix height.

4. The ventilation hose of claim 1, wherein a width of the helix (at a lower side which rests on the continuous wall) ranges from 1.8 mm to 4 mm.

5. The ventilation hose of claim 1, wherein a spacing between two helices corresponds to a pitch of the helix, the pitch ranging from 3 mm to 8 mm.

6. The ventilation hose of claim 1, wherein the helix tapers with increasing distance from the continuous wall, due to which a spacing between two adjacent helices is different at at least three points, and wherein a spacing between two adjacent helices is greatest in an area of a maximum helix height.

7. The ventilation hose of claim 1, wherein the helix surrounds the continuous wall in the form of a helix of 2.7 mm with 5 mm pitch at an internal diameter of Ø15 mm hose.

8. The ventilation hose of claim 1, wherein the continuous wall has a thickness of from 0.05 mm to 0.3 mm.

9. The ventilation hose of claim 1, wherein a compliance of the hose is less than 1.2 ml/hPa/m.

10. The ventilation hose of claim 1, wherein a leakage at a respiratory gas pressure of up to 60 hPa is less than 15 ml/min/m.

11. The ventilation hose of claim 1, wherein a spring rate ranges from 25 N/m or greater than 25 N/m.

12. The ventilation hose of claim 1, wherein an extensibility ranges from 3% to 38%.

13. The ventilation hose of claim 1, wherein the continuous wall is produced from a first silicone material and wherein the helix is produced from a second PVC or TPE material.

14. The ventilation hose of claim 1, wherein the first material and the second material are identical.

15. The ventilation hose of claim 1, wherein a weight per unit of length is less than 1000 g/m.

16. A method for producing a ventilation hose comprising a continuous wall which delimits an internal lumen, the lumen being configured to guide respiratory gas and a helix enclosing the continuous wall in a spiral shape on a side facing away from the lumen, wherein the method comprises producing the continuous wall from a first material and producing the helix from a second material, and connecting the helix to the continuous wall in a co-extrusion process.

17. The method of claim 16, in which in a first method step, the hose is heated above a softening temperature of the material and, in a second method step, the hose is compressed in relation to its original length and, in a third method step, the hose is cooled until a temperature falls below a solidification temperature of the material.

18. The method of claim 16, wherein heating and compression take place in direct succession and a compressed hose is cooled.

19. The method of claim 16, wherein the hose is heated to above 70° C. and compressed on block, so that helices touch one another, and wherein the hose is cooled to a temperature of below 80° before compression is ended.

20. The method of claim 16, wherein the hose is heated from an inside and cooled from the inside.
